(19) United States
(12) Patent Application Publication
(10) Pub. No.: US 2005/0112677 A1
(43) Pub. Date: May 26, 2005

(54) Substrate having oxide layer, method for detecting target material using the substrate, and optical sensor including the substrate

(76) Inventor: Jeo-young Shim, Yongin-si (KR)

Correspondence Address: CANTOR COLBURN, LLP, 55 GRIFFIN ROAD SOUTH, BLOOMFIELD, CT, 06002, US

(21) Appl. No.: 10/994,626

(22) Filed: Nov. 22, 2004

(30) Foreign Application Priority Data
Nov. 22, 2003 (KR) 2003-83356

(51) Int. Cl. C12Q 1/68; G01N 33/53; G01N 33/537; G01N 33/543; C12M 1/34

(52) U.S. Cl. 435/6; 435/7.92; 435/287.2

(57) ABSTRACT

Provided are a substrate used in optically detecting a target material and having an oxide layer, a method for detecting a target material using the substrate, and an optical sensor including the substrate. The substrate can provide an increased detection signal in an analysis method using the substrate.

### BACKGROUND OF THE INVENTION

This application claims priority from Korean Patent Application No. 2003-83356, filed on Nov. 22, 2003, in the Korean Intellectual Property Office, the disclosure of which is incorporated herein in its entirety by reference.

1. Field of the Invention

The present invention relates to a substrate having an oxide layer, a method for detecting a target material using the substrate, and an optical sensor including the substrate.

2. Description of the Related Art

As a conventional biological/chemical analysis technique, it is known a technique for detecting or assaying a target material in which a specific material is immobilized on a substrate and captures the target material by specific bond formation. Examples of such an analysis technique include an enzyme linked immunosorbent assay (ELISA)-based technique and a microarray-based technique. Generally, according to such an analysis technique, a specific compound (commonly referred to as “probe compound”) is immobilized on a substrate and incubated with a sample containing a target material specifically binding with the probe compound. At this time, the target material may be labeled or unlabelled. After the incubation, a specific reaction between the probe compound and the target material is detected by using a detectable signal. The detectable signal may be an optical or electrical signal. In the case of using the optical signal, generally, a reaction product between the probe compound and the target material is illuminated by excitation light and light emitted from the reaction product is measured to thereby detect the presence of the target material.

Recently, a microarray analysis technique is widely used. A microarray is an analysis system in which specific molecules are immobilized in a high density on a substrate. Examples of the microarry include a polynucleotide microarray and a protein microarray. The polynucleotide microarray is an analysis system in which polynucleotide groups are immobilized in a high density on a substrate. The polynucleotide groups are immobilized on predetermined regions of the polynucleotide microarray. Such a microarray is well known in the pertinent art. Examples of the microarray are disclosed in U.S. Pat. Nos. 5,445,934 and 5,744,305. A method of fabricating a microarray using photolithography is generally known. According to a method of fabricating a polynucleotide microarray using photolithography, predetermined regions of a substrate coated with a monomer having a removable protecting group are exposed to an energy source to remove the protecting group. Then, the deprotected monomer is coupled with a monomer having a removable protecting group. Repetition of the above processes produces a polynucleotide microarray. In this case, polynucleotides to be immobilized on the polynucleotide microarray can be prepared by continued extension of polynucleotide monomers. Alternatively, previously synthesized polynucleotides can be immobilized on predetermined regions of the polynucleotide microarray (also called as “spotting technique”). Such fabrication methods for polynucleotide microarrys are illustrated in U.S. Pat. Nos. 5,744,305, 5,143,854, and 5,424,186. The above patent documents about polynucleotide microarrays and fabrication methods thereof are incorporated herein in their entireties by reference.

Generally, a substrate used in a conventional optical analysis technique is surface-untreated or is formed with a grating structure to increase a signal-to-noise ratio. For example, U.S. Pat. No. 6,483,096 discloses a method for increasing a signal-to-noise ratio by separating excitation light and emission light using a circular grating structure.

However, such a conventional technique has problems in that enormous costs are incurred for grating structure formation and a guided emission light to be measured has a relatively low intensity. Therefore, an increase of a signal-to-noise ratio is still being required. In view of these problems, the present inventors found that an oxide layer formed on a substrate can increase the intensity of a signal and completed the present invention.

### SUMMARY OF THE INVENTION

The present invention provides a substrate having an oxide layer.

The present invention also provides a method for detecting a target material using the substrate.

The present invention also provides an optical sensor including the substrate.

According to an aspect of the present invention, there is provided a substrate used in optically detecting a target material and having an oxide layer.

According to another aspect of the present invention, there is provided a method for detecting a target material, which includes: immobilizing a probe material on a substrate used in optically detecting the target material and having an oxide layer; reacting the immobilized probe material and the target material; illuminating a reaction product with excitation light; and measuring light emitted from the reaction product by the excitation light.

According to another aspect of the present invention, there is provided an optical sensor for target material detection including a substrate used in optically detecting a target material and having an oxide layer.

### BRIEF DESCRIPTION OF THE DRAWINGS

FIG. 1 illustrates a substrate having an oxide layer thereon;

FIG. 2 is a graph that illustrates a signal intensity according to the thickness of an oxide layer when gamma-aminopropyltriethoxy silane (GAPS) or gamma-aminopropyldiethoxy silane (GAPDES) is used as a coupling agent;

FIG. 3 illustrates the intensity of a signal emitted from a polynucleotide microarray having an oxide layer with a thickness of 1,000 Å;

FIG. 4 illustrates a detection result of a glyceraldehydes-3-phosphate dehydrogenase (GAPDH) gene sample using a polynucleotide microarray having an oxide layer with a thickness of 1,000 Å; and

FIG. 5 illustrates a detection result of a GAPDH gene sample using a polynucleotide microarray having no oxide layers.

### DETAILED DESCRIPTION OF THE INVENTION

The present invention provides a substrate used in optically detecting a target material and having an oxide layer.

In the present invention, there are no particular limitations on the substrate provided that it can be used in optically detecting a target material. The substrate may be a microarray substrate or an enzyme linked immunosorbent assay (ELISA) substrate, but is not limited thereto. The substrate may be made of a material commonly known in the pertinent art. For example, the substrate may be made of a glass such as borosilicate and boroaluminosilicate, a silicon, or a plastic material such as polyethylene, polypropylene, polyacrylamide, and polystyrene.

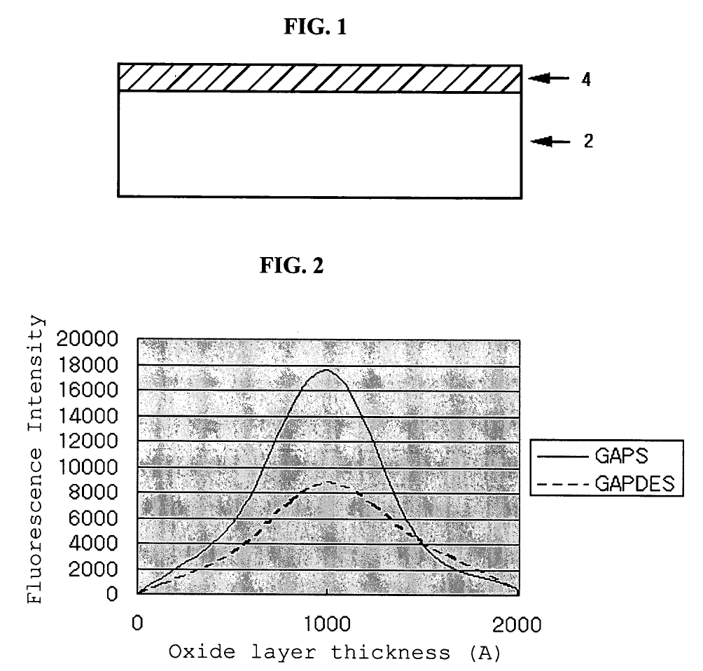

In the present invention, the oxide layer may vary according to the wavelength of excitation light used. Preferably, the oxide layer has a thickness of 500-1,500 Å. Formation of the oxide layer on the substrate can be carried out by methods well known in the pertinent art. According to a method of forming the oxide layer, fused silica (SiO₂) is deposited on a silicon substrate by plasma enhanced chemical vapor deposition (PECVD). According to another method of forming the oxide layer, fused silica (SiO₂) is uniformly spin-coated on a silicon substrate. The oxide layer is used to increase the intensity of excitation light. A mechanism of increasing the intensity of excitation light by the oxide layer can be described by constructive interference that occurs between excitation light reflected from a surface of the oxide layer and excitation light reflected from the substrate after being refracted while passing through the oxide layer, but is not limited thereto. FIG. 1 illustrates an example of a substrate 2 having an oxide layer 4 thereon according to the present invention.

The present invention also provides a method for detecting a target material, which includes:

immobilizing a probe material on the substrate of the present invention;

reacting the target material and the immobilized probe material;

illuminating a reaction product with excitation light; and

measuring light emitted from the reaction product by the excitation light.

In the method of the present invention, the substrate is not particularly limited provided that it can be used in an optical detection method of the target material. As used herein, the term “optical detection method” indicates a method for detecting the target material by converting a specific reaction between the probe material and the target material to an optical signal and measuring the optical signal. The substrate may be a polynucleotide or protein microarray substrate, but is not limited thereto. The “probe material” is a material that is immobilized on the substrate to specifically bind with the target material. For example, when the target material is a polynucleotide, the probe material is a polynucleotide complementarily binding with the target polynucleotide. On the other hand, when the target material is a protein, the probe material is a ligand, an antigen, or an antibody, which specifically binds with the target protein. In the present invention, the target material may be unlabelled or labeled with an optically active material. In the case of the former, the probe material may be labeled with an optical material specifically binding with the target material. The optically active material may be a fluorescent material or a phosphorescent material. The fluorescent material may be fluorescein, Cy-5, or Cy-3.

The present invention also provides an optical sensor for target material detection including the substrate of the present invention. Preferably, the optical sensor includes the substrate of the present invention; a device depositing a target material to be detected; a device illuminating excitation light; and a device detecting light emitted from the target material by the excitation light. Here, the device depositing the target material is well known in the pertinent art, and for example, may be an automatic pipette or suction means that can deposit a small quantity of a sample (nl or μl unit). The device illuminating the excitation light and the device detecting the emission light are respectively an optical source and a detector detecting the emission light which are commonly known in the pertinent art.

Hereinafter, the present invention will be described more specifically by Example. However, the following Example is provided only for illustration and thus the present invention is not limited thereto.

### EXAMPLE 1

In this Example, microarrays were fabricated by forming SiO₂ layers to a thickness of 500-2,000 Å on silicon wafers, followed by linkage with a coupling agent and immobilization of probe polynucleotides. Then, the microarrays were incubated with labeled target nucleic acids and exposed to excitation light, and light emitted from the target nucleic acids was measured, to evaluate the intensity of detected signals with respect to the thickness of the SiO₂ layers.

1. Formation of Oxide Layers on Wafers

Silicon wafers were used. Oxide layers were formed on the silicon wafers by thermal oxidation using Furnace SVF-200 (Celtron). The oxide layers were formed to a thickness of 500-2,000 Å.

The thickness of the oxide layers was measured using NANOSPEC Model AFT 200 (NANOMETTICS). The NANOSPEC Model AFT 200 is equipment that measures the thickness of an oxide layer using the principle that when light enters a silicon wafer, some light is reflected from the oxide layer on the silicon wafer and some light is reflected from the silicon wafer after passing through the oxide layer. The thickness of the oxide layer is measured using a phase difference between the light reflected from the oxide layer and the light reflected from the silicon wafer. In the Example, the silicon wafers were placed on a sample stage of the NANOSPEC and the thickness of the oxide layer at 5-6 points on each silicon wafer was measured to obtain an average thickness. Based on the average thickness, the silicon wafers having a predetermined oxide layer thickness were used in a subsequent coating process. All experiments were performed in a cleanroom-class 1000 with few or no dust particles.

2. Coating with Fluorescent Dye and Evaluation of Fluorescence Intensity with Respect to Thickness of Oxide Layers

First, the silicon wafers were carefully rinsed before surface treatment. The rinsing was performed using pure acetone and water. Then, organic contaminants were removed from the silicon wafers using a piranha solution (a 3:1 mixture of sulfuric acid and hydrogen peroxide). Finally, the silicon wafers were washed with abundant water and acetone and dried. The wafer rinsing process was performed in a wet station used in a semiconductor fabrication process, the piranha solution was made using a sulfuric acid bath, and the washing was performed using a QDR (quick dump rinse) process.

Next, the silicon wafers were fixed to wafer carriers made of Teflon, rinsed, and dried by a spin drier. In detail, immediately after the rinsing process, the silicon wafers were spin-coated with a solution of GAPS (γ-aminopropyltriethoxy silane) (20%, v/v) or GAPDES (γ-aminopropyidiethoxy silane) (20%, v/v) in ethanol. The spin coating was performed using a spin coater model CEE 70 (CEE) in the following manner: initial coating at a rate of 500 rpm/10 sec and main coating at a rate of 2,000 rpm/10 sec. After the spin coating was completed, the silicon wafers were fixed to the wafer carriers made of Teflon and cured at 120° C. for 40 minutes. The cured wafers were immersed in water for 10 minutes, ultrasonic-washed for 15 minutes, immersed in water for 10 minutes, and dried. The drying was performed using a spin-drier. After the drying was completed, the wafers were cut into square or rectangular pieces for subsequent experiments. All experiments were performed in cleanroom-class 1000 with few or no dust particles.

The resulting silane-modified wafers were coated with fluorescein (0.05 g/10 ml). The coating with the fluorescein was performed by immersion. In detail, first, the fluorescein was dissolved in a DMF (dimethylformamide) solution to prepare an immersion solution (0.05 g fluorescein/10 ml). The immersion solution and the silane-modified wafers were placed in a reaction chamber and incubated at 40° C. for 120 minutes. After reaction termination, the silane-modified wafers were removed from the immersion solution and then rinsed. The rinsing was performed with DMF (three times, 10 minutes for each) and methanol (three times, 10 minutes for each). The rinsed wafers were dried and then fluorescein attached to the wafers was quantified using GenePix 4000B scanner (Axon). Scanning was performed by illuminating a 532 nm light and measuring a fluorescence intensity at 570 nm.

According to the quantitative results, when GAPS was used as the coupling agent, the wafers having the oxide layers with thicknesses of 10, 500, 1,000, 1,500, and 2,000 Å produced the fluorescence intensity of 90, 5,600, 17,600, 4,000, and 500 a.u., respectively. Furthermore, when GAPDES was used as the coupling agent, the wafers having the oxide layers with thicknesses of 10, 500, 1,000, 1,500, and 2,000 Å produced the fluorescence intensity of 60, 3,200, 9,000, 4,200, and 600 a.u., respectively, (see FIG. 2). As seen from the above results, the fluorescence intensity was the highest in the wafers having the oxide layers with a thickness of 1,000 Å, which was about 200 times higher than that of wafers having no oxide layers. In addition, the wafers treated with GAPS as the coupling agent provided a higher fluorescence intensity, as compared to the wafers treated with GAPDES.

3. Fabrication of Polynucleotide Microarrays and Evaluation of Fluorescence Intensity with Respect to Thickness of Oxide Layers

Probe polynucleotides were immobilized on the wafers as prepared in Section 2, which had the oxide layers with the thickness of 1,000 Å and had been treated with GAPS, and hybridized with target polynucleotides labeled with Cy-3. Then, a fluorescence intensity was measured at 532 nm.

In detail, the immobilization was performed by spotting a spotting solution containing the probe polynucleotides on the wafers. The spotting solution was prepared by adding the probe polynucleotides in a 100 mM NaHCO₃ (pH 9.0) solution, followed by stirring and incubation at 37° C. for 1 hour. The spotting solution was spotted on the wafers and incubated in a wet chamber, which had been set to 70° C. and 40% of RH (relative humidity), for one hour. Then, amino groups on unspotted surfaces of the wafers were reacted with a blocking agent to be negatively charged (background control) so that the target polynucleotides were not attached to the unspotted surfaces of the wafers, and then the wafers were maintained in a drying machine. The wafers thus prepared, i.e., DNA chips, were incubated with the target polynucleotides labeled with Cy-3 for hybridization, and then, a fluorescence intensity was measured at 532 nm.

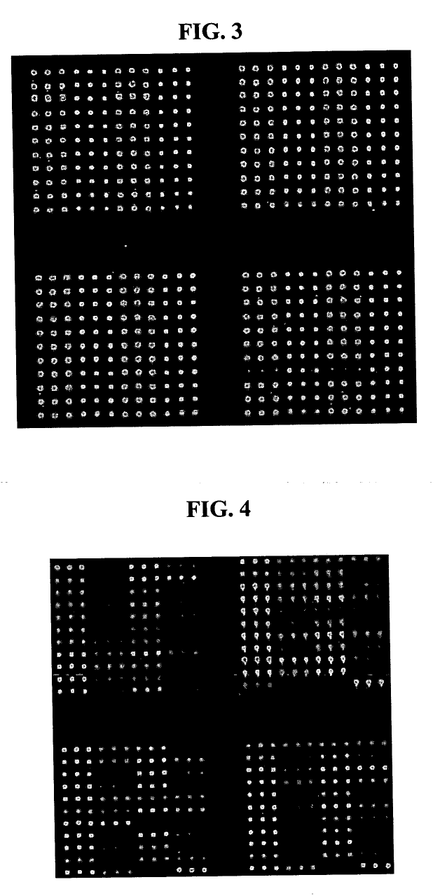

The results are shown in FIGS. 3 and 4. FIG. 3 shows results of hybridization between wild-type and mutant probe polynucleotides as set forth in SEQ ID NOS: 1 and 2 and a target polynucleotide as set forth in SEQ ID NO: 3. In FIG. 3, red spots are perfectly matched hybridization results and yellow spots are mismatched hybridization results.

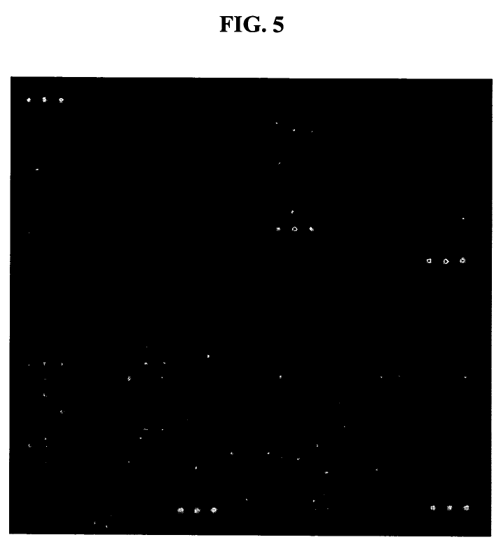

FIG. 4 shows results of hybridization between MODY 3 diabetes-associated probe polynucleotides as set forth in SEQ ID NOS: 4-79 and human glyceraldehyde-3-phosphate dehydrogenase (GAPDH) encoding gene as a target polynucleotide. FIG. 5 shows the hybridization results in polynucleotide microarrays having no oxide layers as control. As shown in FIGS. 4 and 5, the polynucleotide microarrays having wafers with no oxide layers produced a remarkably low signal intensity, as compared to those with oxide layers.

As seen from the Example, when a target polynucleotide is detected using a microarray including a substrate with an oxide layer, a remarkably excellent fluorescent signal can be obtained, as compared to that with no oxide layers.

The substrate having an oxide layer according to the present invention can be efficiently used as a microarray or ELISA substrate.

The method for detecting a target material according to the present invention can provide a high signal-to-noise ratio, thereby ensuring high detection sensitivity.

The optical sensor according to the present invention can be used in detecting a target material with high detection sensitivity.

1. A substrate used in optically detecting a target material and having an oxide layer.

2. The substrate of claim 1, wherein the oxide layer has a thickness of 500-1,500 Å.

3. The substrate of claim 1, wherein the substrate is made of silicon or glass.

4. The substrate of claim 1, wherein the substrate is a microarray substrate or an enzyme linked immunosorbent assay (ELISA) substrate.

5. A method for detecting a target material, which comprises:
immobilizing a probe material on a substrate, wherein the substrate used in optically detecting a target material and having an oxide layer;
reacting the immobilized probe material and the target material;
illuminating a reaction product with excitation light; and
measuring light emitted from the reaction product by the excitation light.

6. The method of claim 5, wherein the target material is labeled with an optically active material.

7. The method of claim 6, wherein the optically active material is a fluorescent or phosphorescent material.

8. An optical sensor for target material detection comprising.
a substrate used in optically detecting a target material and having an oxide layer.

9. The optical sensor of claim 8, further comprising:
a device depositing a target material to be detected;
a device illuminating excitation light; and
a device detecting light emitted from the target material by the excitation light.

10. The optical sensor of claim 8, wherein the substrate is a microarray substrate or an enzyme linked immunosorbent assay (ELISA) substrate.

11. The method of claim 5, wherein the oxide layer has a thickness of 500-1,500 Å.

12. The method of claim 5, wherein the substrate is made of silicon or glass.

13. The method of claim 5, wherein the substrate is a microarray substrate or an enzyme linked immunosorbent assay (ELISA) substrate.


SEQUENCE LISTING (79 sequences)

SEQ ID NO: 1 - DNA, 15 bp, Artificial Sequence
  wild-type probe oligonucleotide
cggaggaaccgtttc

SEQ ID NO: 2 - DNA, 15 bp, Artificial Sequence
  mutant type probe oligonucleotide
cggaggaaccatttc

SEQ ID NO: 3 - DNA, 15 bp, Artificial Sequence
  target oligonucleotide
cggaggaaccgtttc

SEQ ID NO: 4 - DNA, 14 bp, Artificial Sequence
  probe oligonucleotide
cagctggctcagtt

SEQ ID NO: 5 - DNA, 12 bp, Artificial Sequence
  probe oligonucleotide
agctcctggcgg

SEQ ID NO: 6 - DNA, 15 bp, Artificial Sequence
  probe oligonucleotide
gcctggatcagtgcc

SEQ ID NO: 7 - DNA, 13 bp, Artificial Sequence
  probe oligonucleotide
gcactgggtgagc

SEQ ID NO: 8 - DNA, 11 bp, Artificial Sequence
  probe oligonucleotide
gggagtcctgc

SEQ ID NO: 9 - DNA, 15 bp, Artificial Sequence
  probe oligonucleotide
cagaagggtctccac

SEQ ID NO: 10 - DNA, 12 bp, Artificial Sequence
  probe oligonucleotide
ctcccccttgtc

SEQ ID NO: 11 - DNA, 15 bp, Artificial Sequence
  probe oligonucleotide
agaagggtctccacc

SEQ ID NO: 12 - DNA, 12 bp, Artificial Sequence
  probe oligonucleotide
aaggccccctgg

SEQ ID NO: 13 - DNA, 16 bp, Artificial Sequence
  probe oligonucleotide
atggtcaagtcctacc

SEQ ID NO: 14 - DNA, 15 bp, Artificial Sequence
  probe oligonucleotide
caacatcccacagcg

SEQ ID NO: 15 - DNA, 13 bp, Artificial Sequence
  probe oligonucleotide
cgctgtgggatgt

SEQ ID NO: 16 - DNA, 13 bp, Artificial Sequence
  probe oligonucleotide
cctcccgctgtgg

SEQ ID NO: 17 - DNA, 12 bp, Artificial Sequence
  probe oligonucleotide
cacctcccgctg

SEQ ID NO: 18 - DNA, 15 bp, Artificial Sequence
  probe oligonucleotide
ggaggtggtcgatac

SEQ ID NO: 19 - DNA, 14 bp, Artificial Sequence
  probe oligonucleotide
aggtgggactggtt

SEQ ID NO: 20 - DNA, 13 bp, Artificial Sequence
  probe oligonucleotide
accagtcccacct

SEQ ID NO: 21 - DNA, 12 bp, Artificial Sequence
  probe oligonucleotide
gcagaagcgggc

SEQ ID NO: 22 - DNA, 11 bp, Artificial Sequence
  probe oligonucleotide
gaagcgggccg

SEQ ID NO: 23 - DNA, 12 bp, Artificial Sequence
  probe oligonucleotide
cgggccgccctg

SEQ ID NO: 24 - DNA, 16 bp, Artificial Sequence
  probe oligonucleotide
tggtcgataccactgg

SEQ ID NO: 25 - DNA, 14 bp, Artificial Sequence
  probe oligonucleotide
acaagggcactccc

SEQ ID NO: 26 - DNA, 13 bp, Artificial Sequence
  probe oligonucleotide
acttacgctgcgc

SEQ ID NO: 27 - DNA, 12 bp, Artificial Sequence
  probe oligonucleotide
cttcgccacacg

SEQ ID NO: 28 - DNA, 13 bp, Artificial Sequence
  probe oligonucleotide
gtgttgggacagg

SEQ ID NO: 29 - DNA, 12 bp, Artificial Sequence
  probe oligonucleotide
ccaacacctcaa

SEQ ID NO: 30 - DNA, 12 bp, Artificial Sequence
  probe oligonucleotide
cacctgtgggct

SEQ ID NO: 31 - DNA, 12 bp, Artificial Sequence
  probe oligonucleotide
ccgccccttctt

SEQ ID NO: 32 - DNA, 14 bp, Artificial Sequence
  probe oligonucleotide
aaacggttcctccg

SEQ ID NO: 33 - DNA, 15 bp, Artificial Sequence
  probe oligonucleotide
cggaggaaccgtttc

SEQ ID NO: 34 - DNA, 15 bp, Artificial Sequence
  probe oligonucleotide
ccccacttgaaacgg

SEQ ID NO: 35 - DNA, 15 bp, Artificial Sequence
  probe oligonucleotide
ctgcctctcataggc

SEQ ID NO: 36 - DNA, 14 bp, Artificial Sequence
  probe oligonucleotide
aggaggagcgagag

SEQ ID NO: 37 - DNA, 13 bp, Artificial Sequence
  probe oligonucleotide
agcgagagacgct

SEQ ID NO: 38 - DNA, 13 bp, Artificial Sequence
  probe oligonucleotide
atgctgggcccca

SEQ ID NO: 39 - DNA, 13 bp, Artificial Sequence
  probe oligonucleotide
agcgagagacgct

SEQ ID NO: 40 - DNA, 15 bp, Artificial Sequence
  probe oligonucleotide
ctcctccactagcgt

SEQ ID NO: 41 - DNA, 14 bp, Artificial Sequence
  probe oligonucleotide
ctggatgcattccg

SEQ ID NO: 42 - DNA, 15 bp, Artificial Sequence
  probe oligonucleotide
gcggaatgcatccag

SEQ ID NO: 43 - DNA, 11 bp, Artificial Sequence
  probe oligonucleotide
gcccagcccct

SEQ ID NO: 44 - DNA, 14 bp, Artificial Sequence
  probe oligonucleotide
acctcgtcacggag

SEQ ID NO: 45 - DNA, 12 bp, Artificial Sequence
  probe oligonucleotide
ctccgtgacgag

SEQ ID NO: 46 - DNA, 13 bp, Artificial Sequence
  probe oligonucleotide
cacgcacctccgt

SEQ ID NO: 47 - DNA, 12 bp, Artificial Sequence
  probe oligonucleotide
cacgcacctccg

SEQ ID NO: 48 - DNA, 15 bp, Artificial Sequence
  probe oligonucleotide
gaggtgcgtgtctac

SEQ ID NO: 49 - DNA, 13 bp, Artificial Sequence
  probe oligonucleotide
accggcgcaaaga

SEQ ID NO: 50 - DNA, 12 bp, Artificial Sequence
  probe oligonucleotide
cccccagggcca

SEQ ID NO: 51 - DNA, 13 bp, Artificial Sequence
  probe oligonucleotide
cccccccagggcc

SEQ ID NO: 52 - DNA, 15 bp, Artificial Sequence
  probe oligonucleotide
tggcaaaccagttgt

SEQ ID NO: 53 - DNA, 16 bp, Artificial Sequence
  probe oligonucleotide
caactggtttgccaac

SEQ ID NO: 54 - DNA, 13 bp, Artificial Sequence
  probe oligonucleotide
gcccgctcacagc

SEQ ID NO: 55 - DNA, 15 bp, Artificial Sequence
  probe oligonucleotide
cccccagtaaggtcc

SEQ ID NO: 56 - DNA, 12 bp, Artificial Sequence
  probe oligonucleotide
gggcggaatgca

SEQ ID NO: 57 - DNA, 12 bp, Artificial Sequence
  probe oligonucleotide
ccccacgggcct

SEQ ID NO: 58 - DNA, 14 bp, Artificial Sequence
  probe oligonucleotide
tcccccctgtcagc

SEQ ID NO: 59 - DNA, 14 bp, Artificial Sequence
  probe oligonucleotide
tcccccctgtcagc

SEQ ID NO: 60 - DNA, 14 bp, Artificial Sequence
  probe oligonucleotide
cagacatccccagg

SEQ ID NO: 61 - DNA, 12 bp, Artificial Sequence
  probe oligonucleotide
accatcgggcct

SEQ ID NO: 62 - DNA, 14 bp, Artificial Sequence
  probe oligonucleotide
accagggtggaggc

SEQ ID NO: 63 - DNA, 14 bp, Artificial Sequence
  probe oligonucleotide
cgcaggcacagagt

SEQ ID NO: 64 - DNA, 11 bp, Artificial Sequence
  probe oligonucleotide
gaccggcacac

SEQ ID NO: 65 - DNA, 12 bp, Artificial Sequence
  probe oligonucleotide
acccagaacccc

SEQ ID NO: 66 - DNA, 12 bp, Artificial Sequence
  probe oligonucleotide
gagcggctgctg

SEQ ID NO: 67 - DNA, 13 bp, Artificial Sequence
  probe oligonucleotide
tgggcgtgaggct

SEQ ID NO: 68 - DNA, 13 bp, Artificial Sequence
  probe oligonucleotide
acgcccaccaagc

SEQ ID NO: 69 - DNA, 14 bp, Artificial Sequence
  probe oligonucleotide
agacactgaggcct

SEQ ID NO: 70 - DNA, 13 bp, Artificial Sequence
  probe oligonucleotide
cggcatctcaggc

SEQ ID NO: 71 - DNA, 12 bp, Artificial Sequence
  probe oligonucleotide
ctgccggcatcc

SEQ ID NO: 72 - DNA, 13 bp, Artificial Sequence
  probe oligonucleotide
ccggcccaccggc

SEQ ID NO: 73 - DNA, 13 bp, Artificial Sequence
  probe oligonucleotide
cccaccggctcag

SEQ ID NO: 74 - DNA, 12 bp, Artificial Sequence
  probe oligonucleotide
ccggctcagcgc

SEQ ID NO: 75 - DNA, 15 bp, Artificial Sequence
  probe oligonucleotide
ccccacaggtgagag

SEQ ID NO: 76 - DNA, 13 bp, Artificial Sequence
  probe oligonucleotide
tctcgatgacgct

SEQ ID NO: 77 - DNA, 14 bp, Artificial Sequence
  probe oligonucleotide
gagatgaaggtctc

SEQ ID NO: 78 - DNA, 18 bp, Artificial Sequence
  probe oligonucleotide
tctgtttacattggagct

SEQ ID NO: 79 - DNA, 13 bp, Artificial Sequence
  probe oligonucleotide
ttgggggggcagt